(19) United States
(12) Patent Application Publication
(10) Pub. No.: US 2026/0168038 A1
(43) Pub. Date: Jun. 18, 2026

(54) SYSTEMS AND METHODS FOR ENDOSCOPIC NAVIGATION AND BOOKMARKING

(71) Applicant: Verily Life Sciences LLC, Dallas, TX (US)

(72) Inventors: Kirk Gossage, Pacifica, CA (US); Grant Anderson, Hayward, CA (US)

(21) Appl. No.: 19/113,660

(22) PCT Filed: Oct. 11, 2023
(86) PCT No.: PCT/US2023/034913

§ 371 Date: Mar. 20, 2025

Related U.S. Application Data
(60) Provisional application No. 63/380,608, filed on Oct. 24, 2022.

(51) Int. Cl. C12Q 1/6895; A61K 38/02; A63B 53/04; B82Y 20/00; C07K 16/18; E02D 7/02; F21K 9/60; G06F 8/38; G06F 16/955; G06Q 10/10; G06Q 10/40; G06Q 40/04; G06Q 40/08; G09B 23/28; H04B 7/26; H04J 1/02; H04L 9/40; H04W 48/08; H10H 20/853; H10W 20/00

(52) U.S. Cl. CPC C12Q 1/6895; A61K 38/02; A63B 53/045; B82Y 20/00; C07K 16/18; E02D 7/02; F21K 9/60; G06F 8/38; G06F 16/9566; G06Q 10/10; G06Q 10/40; G06Q 40/04; G06Q 40/08; G09B 23/28; H04B 7/2606; H04J 1/02; H04L 63/0838; H04W 48/08; H10H 20/853; H10W 20/033; A61M 2210/0693; B60C 2200/10; B65H 2801/31; C07K 2317/52; D05D 2209/10; H01H 2239/032; H04L 2209/56; H04Q 2213/13076

(57) ABSTRACT

Systems and methods for determining a position of an endoscope in a portion of a body are described. In an embodiment, the system includes an endoscope comprising an endoscope body shaped to enter a portion of a body, the endoscope body defining a proximal end and a distal end opposite the proximal end; an insertion depth sensor configured to generate an insertion depth signal based on an insertion depth of the endoscope body within the portion of the body; and an inertial measurement unit disposed at the distal end of the endoscope body, the inertial measurement unit configured to generate an orientation signal based upon an orientation of the inertial measurement unit. In an embodiment, the system is configured to generate positional information of the distal end of the endoscope body within the portion of the body based upon the insertion depth signal and the orientation signal.

### CROSS-REFERENCE TO RELATED APPLICATIONS

This application claims the benefit of U.S. Provisional Application 63/380,608, filed Oct. 24, 2022, which is hereby incorporated by reference in its entirety.

### TECHNICAL FIELD

This disclosure relates generally to systems, apparatuses, and methods for endoscopic navigation, and, in particular but not exclusively, relates to endoscopic mapping and bookmarking.

### BACKGROUND INFORMATION

Navigating an interior portion of a body can be difficult with conventional endoscopic systems and methods. Using such conventional systems and methods, operators may know a beginning point and an end point, such as in terms of an insertion depth, but between these two points there is typically uncertainty and estimation regarding a position of the endoscope within the portion of the body. Further, many such conventional endoscopic systems and methods do not automatically measure an insertion depth.

Accordingly, there is presently a need for endoscopic systems and methods suitable to determine where an endoscope is throughout a procedure. Such capabilities would assist with accurate determinations of withdrawal times, accurate positional book marking of portions of interest, coverage estimation, and the like.

### BRIEF DESCRIPTION OF THE DRAWINGS

Non-limiting and non-exhaustive embodiments of the claimed subject matter are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A is a schematic illustration of a system according to an embodiment of the present disclosure.

FIG. 1B is another illustration of the system of FIG. 1A shown within a portion of a body, according to an embodiment of the present disclosure.

FIG. 2A is a cross-sectional view of an insertion depth sensor of a system according to an embodiment of the present disclosure.

FIG. 2B is another cross-sectional view of the insertion depth sensor of FIG. 2A according to an embodiment of the present disclosure.

FIG. 3 is a schematic illustration of a system according to an embodiment of the present disclosure.

FIG. 4 is a block diagram of a process according to an embodiment of the present disclosure.

### DETAILED DESCRIPTION

Embodiments of a system, an apparatus, and a method for endoscopic navigation and bookmarking are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Some portions of the detailed description that follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “selecting”, “identifying”, “capturing”, “adjusting”, “analyzing”, “determining”, “estimating”, “generating”, “comparing”, “modifying”, “receiving”, “providing”, “displaying”, “interpolating”, “outputting”, or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such as information storage, transmission, or display devices.

The algorithms and displays presented herein are not inherently related to any particular computer or other apparatus. Various general-purpose systems can be used with programs in accordance with the teachings herein, or it can prove convenient to construct a more specialized apparatus to perform the required method steps. The required structure for a variety of these systems will appear from the description below. In addition, embodiments of the present disclosure are not described with reference to any particular programming language. It will be appreciated that a variety of programming languages can be used to implement the teachings of the disclosure as described herein.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Conventional endoscopic systems and methods are limited in their ability to map a portion of a body examined or otherwise determine a position of the endoscope within the body. Certain conventional endoscopes have a “ruler-style” visual scale marked on the side for the operator to read by eye. Such conventional endoscopes require a user to determine, for example, a depth of insertion and do not do so automatically. Furthermore, such conventional endoscopes cannot without more determine a position of, for example, a distal end of the endoscope within the body. A path through a portion of a body may not be a straight line and, therefore, a position of the endoscope requires more than simply a depth of insertion.

In various aspects, the present disclosure provides endoscopic systems and methods for endoscopic navigation and bookmarking to address these and related challenges.

Accordingly, in an aspect, the present disclosure provides a system for endoscopic navigation, mapping, and/or bookmarking. In an embodiment, the system comprises an endoscope comprising an endoscope body shaped to enter a portion of a body, the endoscope body defining a proximal end and a distal end opposite the proximal end; an insertion depth sensor configured to generate an insertion depth signal based on an insertion depth of the endoscope body within the portion of the body; and an inertial measurement unit disposed at the distal end of the endoscope body, the inertial measurement unit configured to generate an orientation signal based upon an orientation of the inertial measurement unit.

As discussed further herein, in an embodiment, the insertion depth sensor enables insertion depth information to be automatically known by the system. Further, by matching an insertion depth, such as in real time, with an orientation of the inertial measurement unit, and correspondingly the distal end of the endoscope, a position of the distal end of the endoscope can be determined. As an example, a path through an intestine is not a straight line, but as an endoscope according to an embodiment of the present disclosure is inserted in the colon, it will snake around and progress in the direction that the tip of the endoscope is pointing. By mapping the tip angle and distance inserted, the system can reconstruct a path, such as a three-dimensional path, that the endoscope takes through the colon. This allows for accurate, automated withdrawal time calculations, as well as allow for accurate positional bookmarking of regions of interest within the body, such as polyps in the case of a colonoscopy.

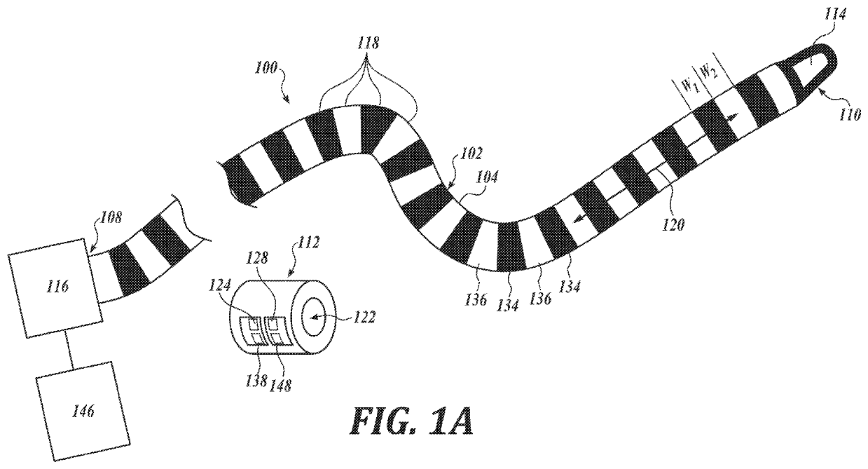

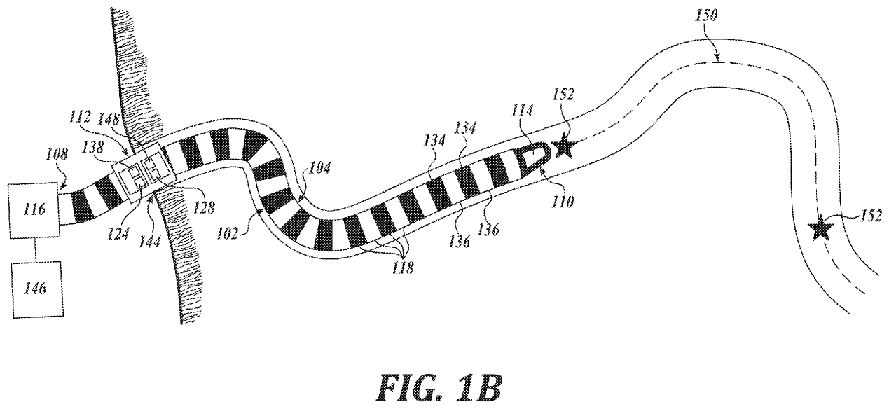

In this regard, attention is now directed to FIGS. 1A and 1B, which illustrate a system 100 according to an embodiment of the present disclosure. FIG. 1A is a schematic illustration of the system 100. FIG. 1B is an illustration of the system 100 shown within a portion of a body 106.

In the illustrated embodiment, the system 100 is shown to include an endoscope 102 including an endoscope body 104, an insertion depth sensor 112, and an inertial measurement unit 114. As shown, the endoscope body 104 is shaped to enter a portion of a body 106 and defines a proximal end 108 and a distal end 110 opposite the proximal end 108. In an embodiment, the endoscope 102 is configured to enter various portions of the body including but not limited to the bladder, the kidney, the bronchus, joints, the colon, the abdomen, and the pelvis. In an embodiment, the endoscope 102 is a device selected from the group consisting of a cystoscope, a nephroscope, a bronchoscope, an arthroscope, a colonoscope, and laparoscope. While various portions of the body, such as the colon, and types of endoscopes are described, such as a colonoscope, it will be understood that the systems and methods of the present disclosure are agnostic to a particular portion of the body and particular types of endoscopes.

Further, the inertial measurement unit 114 is shown disposed at the distal end 110 of the endoscope body 104. The inertial measurement unit 114 is configured measure an orientation of the inertial measurement unit 114, such as by generating an orientation signal based upon an orientation of the inertial measurement unit 114. In an embodiment, the inertial measurement unit 114 generates orientation signals over time, such as with a time stamp or other indication of when the orientation signals are generated. As discussed further herein, by analyzing a depth of insertion and an orientation of the inertial measurement unit 114, and through inference the distal end 110 of the endoscope body 104, a position of the distal end 110 of the endoscope body 104 and/or path 150 of the distal end 110 can be determined, such as in generating a three-dimensional map of a path 150 of the endoscope 102 through the portion of the body 106.

In an embodiment, the inertial measurement unit 114 is a six degree-of-freedom inertial measurement unit 114. In an embodiment, the inertial measurement unit 114 is a nine degree-of-freedom inertial measurement unit 114.

While an inertial measurement unit 114 is described in detail, it will be understood that other sensors configured to measure or generate one or more signals based on an orientation of the distal end 110 of the endoscope body 104 are within the scope of the present disclosure.

As above, the endoscope 102 is shown to include an insertion depth sensor 112. The insertion depth sensor 112 is configured to measure an insertion depth of the endoscope body 104, such as to automatically measure the insertion depth. In an embodiment, insertion depth sensor 112 is configured to generate an insertion depth signal, such as automatically upon insertion into the portion of the body 106, based on an insertion depth of the endoscope body 104 within the portion of the body 106.

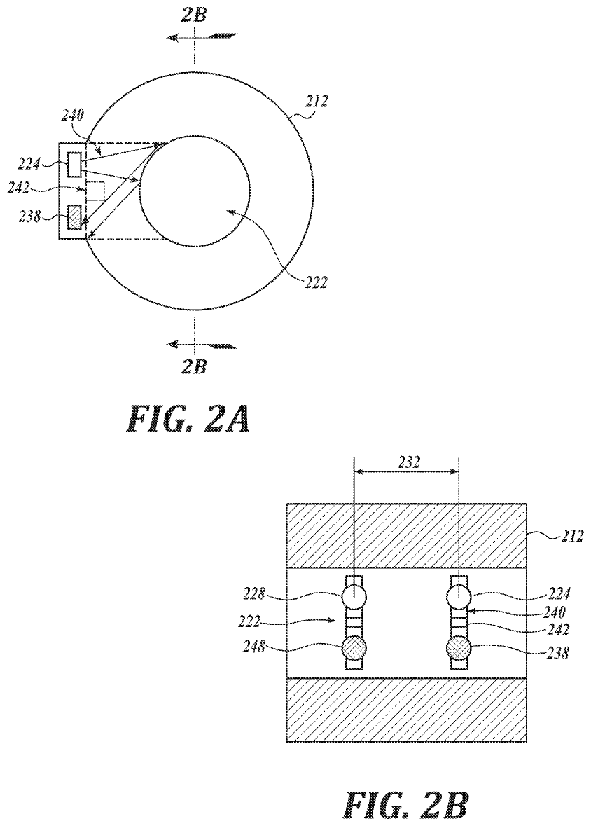

In an embodiment, the insertion depth sensor 112 is integrated as part of or disposed on the endoscope body 104. In the illustrated embodiment, the insertion depth sensor 112 is shaped to couple with or receive the endoscope body 104. The insertion depth sensor 112 is shown to include a first sensor 124 and a second sensor 128, as well as a first light source 138 and a second light source 148, which are discussed further herein with respect to FIGS. 2A and 2B.

As shown, the insertion depth sensor 112 defines an aperture 122 shaped to receive the endoscope body 104. In this regard and as described further herein, as the endoscope body 104 passes through the aperture 122 the insertion depth sensor 112 measures the insertion depth of the endoscope body 104, such as where the insertion depth sensor 112 is positioned at an insertion point 144 in the portion of the body 106. In an embodiment, the insertion depth sensor 112 is shaped to remain at an insertion point 144 in the portion of the body 106 and allow the endoscope body 104 to pass through the aperture 122 into and out of the portion of the portion of the body 106. By remaining at the insertion point 144, the insertion depth sensor 112 can serve as a reference point for the insertion depth. In an embodiment, the insertion depth sensor 112 is a trocar, such as a rectal trocar.

In the illustrated embodiment, the endoscope body 104 defines alternating markings 118, such as alternating markings 118 disposed on the endoscope body 104. As shown, the alternating markings 118 are rotationally invariant about the endoscope body 104. That is to say that the alternating markings 118 do not vary about a longitudinal axis 120 of the endoscope body 104. In an embodiment, the alternating markings 118 comprise a plurality of first markings 134 and a plurality of second markings 136, wherein second markings of the plurality of second markings 136 are interspersed between first markings of the plurality of first markings 134.

In an embodiment, the alternating markings 118 have different colors, such as alternating markings 118 having alternating different colors that can be sensed or measured, such as optically, by the insertion depth sensor 112. Accordingly, in an embodiment, the alternating markings 118 comprise a plurality of first bands 134 having a first color; and a plurality of second bands 136 having a second color different than the first color, wherein second bands of the plurality of second bands 136 are interspersed between first bands of the plurality of first bands 134.

Where alternating markings 118 of different alternating colors are used, in an embodiment, the insertion depth sensor 112 comprises one or more optical sensors used to sense or measure the alternating markings 118 to determine an insertion depth.

In an embodiment, the alternating markings have equal widths, suchas where W₁ is equal to W₂, along a longitudinal axis of the endoscope body. As will now be discussed with respect to FIGS. 2A and 2B, such equal-width alternating markings can be used to determine an insertion depth, such as with appropriately spaced sensors.

FIG. 2A is a cross-sectional view of an insertion depth sensor 212. FIG. 2B is another cross-sectional view of the insertion depth sensor 212. In an embodiment, insertion depth sensor 212 is an example of insertion depth sensor 212 discussed further herein with respect to FIGS. 1A and 1B.

As shown, the insertion depth sensor 212 includes a first sensor 224 and a second sensor 228. In an embodiment, the first sensor 224 configured to generate a first insertion depth signal based on a first portion of an endoscope body, such as endoscope body 104, adjacent to the first sensor 224; and the second sensor 228 configured to generate a second insertion depth signal based on a second portion of the endoscope body adjacent to the second sensor 228. In other words, in an embodiment, the first sensor 224 is positioned in the insertion depth sensor 212 to image, measure, or otherwise sense a portion of the endoscope body when the endoscope body has been inserted within the insertion depth sensor. Likewise, in an embodiment, the second sensor 228 is positioned in the insertion depth sensor 212 to image, measure, or otherwise sense a second portion of the endoscope body when the endoscope body has been inserted in the insertion depth sensor 212, wherein the second portion is different than the first portion.

In an embodiment, a distance 232 between the first sensor 224 and the second sensor 228 is n/2 times the width, such as W₁ and W₂, of the alternating markings, and wherein n is an integer. In an embodiment, n is selected from integers including 1, 2, 3, 4, 5, 6, 7, 8, 9, 10, and the like.

By spacing the first sensor 224 and the second sensor 228 a distance 232 that is n/2 times the width, such as W₁ and W₂, of the equal-width alternating markings, signals from the first sensor 224 and the second sensor 228 are out of phase, such as 90° out of phase. Such out-of-phase signals can be used as a linear quadrature encoder to determine insertion depth.

In an embodiment, generating the first insertion depth signal and the second insertion depth signal includes generating corresponding and/or contemporaneous time stamps associated with the first insertion depth signal and the second insertion depth signal, which may be correlated with time stamps associated with the orientation signal. As discussed further herein, correlating the orientation signal and the insertion depth signal, such as over time, may be used to generate positional information and develop a three-dimensional map or path of the endoscope, and, in particular, the distal end of the endoscope body.

In an embodiment, the insertion depth sensor 212 includes one or more light sources, such as one or more light sources positioned to illuminate the endoscope body, for example, as the endoscope body passes through the aperture 222 of the insertion depth sensor 212. In the illustrated embodiment, the insertion depth sensor 212 includes the first insertion depth sensor 224 and the second insertion depth sensor 228. In an embodiment, the first sensor 224 is a first optical sensor 224 positioned to receive light scattered or reflected from a first portion of the endoscope body; and the second sensor 228 is a second optical sensor 228 positioned to receive light scattered or reflected from a second portion of the endoscope body.

In an embodiment, the insertion depth sensor 212 includes light sources positioned to illuminate the first and/or second portions of the endoscope body, such as when the endoscope body passes through the aperture 222 of the insertion depth sensor 212. In this regard, in an embodiment, the insertion depth sensor 212 includes a first light source 238 positioned to emit first light onto the first portion of the endoscope body. Further, in an embodiment, the insertion depth sensor 212 is shown to include a second light source 248 positioned to emit second light onto the second portion of the endoscope body.

As shown, the insertion depth sensor 212 defines a slit 240 shaped to allow the first light to pass through the insertion depth sensor 212 to the first portion of the endoscope body. The slit 240 is also shaped and positioned to allow first light reflected or scattered off the first portion of the endoscope body to be received by the first sensor 224. The insertion depth sensor 212 is also shown to define a second slit defining analogous features for the second light source 248 and the second sensor 228.

In the illustrated embodiment, the insertion depth sensor 212 comprises a light baffle 242 positioned between the first sensor 224 and the first light source 238. As shown, the light baffle 242 does not extend so that it contacts an endoscope body received by the insertion depth sensor 212. In this way, the light baffle 242 allows light to be reflected or scattered off the endoscope body and received by the sensor 224, but not directly received by the light sensor 224 from the light source 238. In an embodiment, the light baffle 242 is configured to block or limit the first light from passing directly (i.e., without reflecting or scattering off the endoscope body) from the first light source 238 to the first sensor 224. By blocking or limiting un-reflected or un-scattered from reaching the first sensor 224 and the second sensor 228, a higher percentage of scattered or reflected light reaches the first sensor 224 and the second sensor 228, thus increasing a signal-to-noise ratio and improving system performance.

While optical insertion depth sensors are described, it will be understood that other types of sensors and other sensing modalities are within the scope of the present disclosure. As an example, in an embodiment, the insertion depth sensor 112 is shaped or otherwise configured to couple physically and cooperatively to the endoscope body 104 and to generate an insertion depth signal as the endoscope body 104 passes into the portion of the body 106.

As another example, magnetic sensors are possible. Referring again to FIGS. 1A and 1B, in an embodiment, the first sensor 124 and the second sensor 128 are magnetic sensors positioned to generate insertion depth signals based on a magnetic field or a magnetic dipole moment. In an embodiment, the first sensor 124 is a first magnetic sensor positioned to generate insertion depth signals based on a magnetic field or a magnetic dipole moment of a first portion of the endoscope body 104; and the second sensor 128 is a second magnetic sensor positioned to generate insertion depth signals based on a magnetic field or a magnetic dipole moment a second portion of the endoscope body 104. Correspondingly, in an embodiment, the endoscope body 104 includes alternating markings 118 comprising a plurality of first bands having a first magnetic polarity; and a plurality of second bands interspersed between first bands of the plurality of first bands, wherein the plurality of second bands has a second magnetic polarity different than, such as opposite to, the first magnetic polarity.

As above, in an embodiment, the insertion depth sensor is shaped to receive the endoscope body, such as to sense or measure an insertion depth. In an alternative embodiment, however, the insertion depth sensor is integrated in or disposed on the endoscope body.

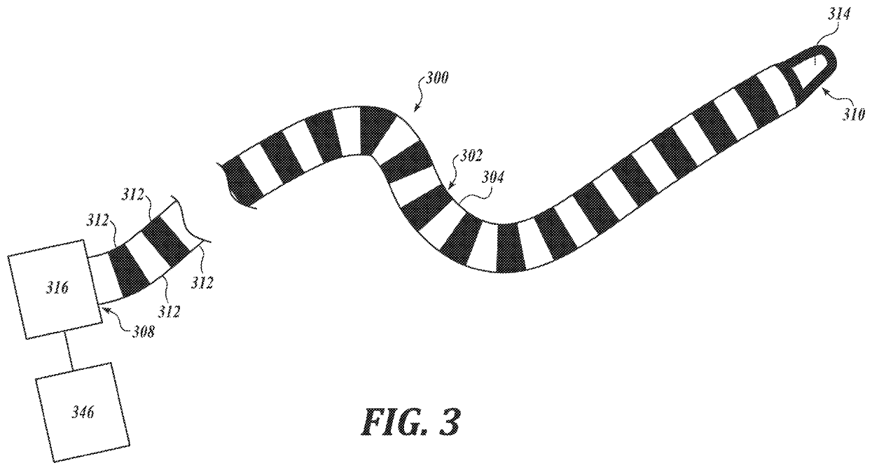

In this regard, attention is directed to FIG. 3, which is a schematic illustration of a system 300. As shown, the system 300 includes an endoscope 302 comprising an endoscope body 304 shaped to enter a portion of a body, the endoscope body 304 defining a proximal end 308 and a distal end 310 opposite the proximal end 308; an insertion depth sensor 312 configured to generate an insertion depth signal based on an insertion depth of the endoscope body 304 within the portion of the body; and an inertial measurement unit 314 disposed at the distal end 310 of the endoscope body 304, the inertial measurement unit 314 configured to generate an orientation signal based upon an orientation of the inertial measurement unit 314.

In the illustrated embodiment, the insertion depth sensor 312 includes one or more sensors disposed on an outer surface of the endoscope body 304. In such an embodiment, the insertion depth sensor 312 can include a plurality of capacitive sensors disposed along the endoscope body 304, wherein a capacitive sensor of the plurality of capacitive sensors is configured to generate a capacitive insertion depth signal in response to contact, such when the capacitive sensor is inserted within and in contact with the portion of the body. By sensing contact between the portion of the body and capacitive sensors of the plurality of capacitive sensors it can be determined which capacitive sensors are in contact with the portion of the body and further determined how far the endoscope 302 is inserted into the portion of the body based on a known position of the capacitive sensors of the plurality of capacitive sensors along the endoscope body 304.

Such an insertion depth can be used with orientation signals from the inertial measurement unit 314 to determine a position or path of the distal end 310 of the endoscope body 304 as discussed elsewhere herein. Further, such capacitive insertion depth signals, as well as orientation signals from the inertial measurement unit 314, can be received by the controller 316 and manipulated or viewed using a user interface 346 operatively coupled to the controller 316.

In an embodiment, the systems of the present disclosure include a controller operatively coupled to various system components to choreograph their operation. Referring again to FIGS. 1A and 1B, the system 100 is shown to include a controller 116 operatively coupled to the insertion depth sensor 112 and the inertial measurement unit 114. In an embodiment, the insertion depth sensor 112 and/or the inertial measurement unit 114 are operatively coupled to the controller 116 through wired connections, although one or more wireless connections are possible and within the scope of the present disclosure. In an embodiment, the controller 116 includes logic that, when executed, causes the system 100 to perform operations including generating positional information of the distal end 110 of the endoscope body 104 within the portion of the body 106 based upon the insertion depth signal and the orientation signal.

In an embodiment, positional information of the distal end 110 of the endoscope body 104 within the portion of the body 106 can be generated over time. In this regard, an insertion depth, and an orientation of the distal end 110 of the endoscope body 104 can be recorded over time and be used to generate a path 150, such as a three-dimensional path 150, through the portion of the body 106. Accordingly, in an embodiment, generating positional information of the distal end 110 of the endoscope body 104 within the portion of the body 106 comprises generating a three-dimensional map of the portion of the body 106 based on a path 150 the distal end 110 has travelled within the portion of the body 106.

As shown, the system 100 further includes a user interface 146 configured to receive input from a user, such as to annotate the three-dimensional map based on the input from the user. In use, a user may annotate a three-dimensional map or other representation of the portion of the body 106 with the user interface 146, shown here with annotation point 152. Such an annotation 152 allows a user to note an aspect or position of a region of interest of the portion of the body 106, such as for later inspection, excision, and the like. As an example, a user may annotate a region of a colon including or thought to include polyps so that such polyps may be noted, further investigated, biopsied, or removed.

In an embodiment, the user interface 146 is a desktop computer, a laptop computer, a tablet, a smartphone, a touch screen, or interface suitable to receive a user input and generate a signal therefrom. As shown, the user interface 146 is operatively coupled to the controller 116, shown here as a wired connection. While a wired connection is shown, it will be understood that a wireless connection is possible and within the scope of the present disclosure.

In an embodiment, the controller 116 includes logic that, when executed, causes the system 100 to perform operations including generating a marker signal when the distal end 110 is located in a portion of the path 150 corresponding to an annotated portion 152 of the three-dimensional map. As above, a user may annotate the path 150 or three-dimensional map, such as for later inspection. In an embodiment, the system 100 generates a marker signal to indicate that the distal end 110 of the endoscope body 104 has returned to the region of interest so that the user may investigate the region further, remove a portion thereof, such as for a biopsy, and the like.

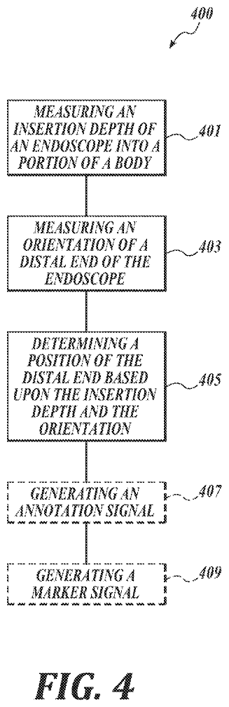

In another aspect, the present disclosure provides a method of determining a position of an endoscope, such as a distal end of an endoscope, within a portion of a body. In this regard, attention is directed to FIG. 4, which is a block diagram of a process according to an embodiment of the present disclosure. The operations of example process 400 are illustrated in order, but operations can be omitted, reordered, repeated, and/or executed in parallel. Operations making up example process 400 can be encoded in computer-readable instructions, as part of a computer-implemented method or as stored on a computer readable memory device.

In an embodiment, process 400 is an example of a method of operating a system according to an embodiment of the present disclosure, such as system 100 discussed further herein with respect to FIGS. 1A and 1B, such as including insertion depth sensor 212 discussed further herein with respect to FIGS. 2A and 2B, or system 300 discussed further herein with respect FIG. 3.

In an embodiment, process 400 begins with process block 401, which includes measuring an insertion depth of the endoscope in the portion of the body. As discussed further herein, measuring an insertion depth can include various measurement modalities and methods, including but not limited to optical, capacitive, resistive, magnetic, mechanical, and the like, such as with one or more insertion depth sensors described herein. In an embodiment, measuring the insertion depth of the endoscope in the portion of the body comprises generating, with an insertion depth sensor, an insertion depth signal based on the insertion depth of the endoscope within the portion of the body. In an embodiment, measuring an insertion depth includes the use of a linear quadrature encoder, such as described further herein with respect to FIGS. 1A, 1B, 2A, and 2B.

In an embodiment, process block 401 is followed by process block 403, which includes measuring an orientation of the distal end of the endoscope. In an embodiment, measuring the orientation includes measuring the orientation of the distal end of the endoscope with an inertial measurement unit. In an embodiment, measuring the orientation of the distal end of the endoscope comprises generating, with an inertial measurement unit disposed at the distal end of the endoscope, an orientation signal based upon an orientation of the inertial measurement unit.

In an embodiment, process block 403 is followed by process block 405, which includes determining the position of the distal end of the endoscope in the portion of the body based upon the measured insertion depth of the endoscope and the measured orientation of the distal end of the endoscope. By measuring the insertion depth of the endoscope body and the orientation of the distal end of the endoscope over time a path of the distal end of the endoscope body can be determined, which can be included in the positional information. In an embodiment, generating positional information of the distal end of the endoscope body within the portion of the body comprises generating a map, such as a three-dimensional map, of the portion of the body based on a path the distal end has travelled within the portion of the body.

In an embodiment, process block 405 is followed by process block 407, which includes generating an annotation signal based on a user input received from a user interface to annotate a portion of the three-dimensional map. In this regard, a user can highlight or mark a portion of the map corresponding to a point or points on the path taken by the distal end of the endoscope, such as for later inspection, further analysis, or excision. In an embodiment, process block 407 is optional.

In an embodiment, process block 407 is followed by process block 409, which includes generating a marker signal when the distal end is located in the portion of the path corresponding to the annotated portion of the three-dimensional map. If the distal end of the endoscope body returns to a portion of the body corresponding to the annotated portion of the map, such as when withdrawing the endoscope from the portion of the body along the path, it may be helpful to alert a user who annotated the map in hopes of viewing the portion of the body, such as for more detailed analysis, sample/biopsy collection, or excision. In an embodiment, the marker signal is displayed on the user interface. In an embodiment, the marker signal includes one or more of an audible sound, a haptic signal, a visual signal, such as a flashing light, and the like.

The order in which some or all of the processes appear in each process should not be deemed limiting. Rather, one of ordinary skill in the art having the benefit of the present disclosure will understand that some of the process blocks may be executed in a variety of orders not illustrated, or even in parallel.

The operations explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (ASIC) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

1. A system comprising:
an endoscope comprising:
an endoscope body shaped to enter a portion of a body, the endoscope body defining a proximal end and a distal end opposite the proximal end;
an insertion depth sensor configured to generate an insertion depth signal based on an insertion depth of the endoscope body within the portion of the body; and
an inertial measurement unit disposed at the distal end of the endoscope body, the inertial measurement unit configured to generate an orientation signal based upon an orientation of the inertial measurement unit; and
a controller operatively coupled to the insertion depth sensor and the inertial measurement unit, the controller including logic that, when executed, causes the system to perform operations including:
generating positional information of the distal end of the endoscope body within the portion of the body based upon the insertion depth signal and the orientation signal.

2. The system of claim 1, further comprising:
alternating markings disposed on the endoscope body, wherein the alternating markings are rotationally invariant about the endoscope body;
wherein the alternating markings have equal widths along a longitudinal axis of the endoscope body.

3. The system of claim 1, wherein the insertion depth sensor defines an aperture shaped to receive the endoscope body.

4. The system of claim 2, wherein the insertion depth sensor comprises a quadrature encoder.

5. The system of claim 4, wherein the first sensor is a first optical sensor positioned to receive light scattered or reflected from the first portion of the endoscope body;
wherein the second sensor is a second optical sensor positioned to receive light scattered or reflected from the second portion of the endoscope body; and
wherein the alternating markings comprise:
a plurality of first bands having a first color; and
a plurality of second bands having a second color different than the first color,
wherein second bands of the plurality of second bands are interspersed between first bands of the plurality of first bands.

6. The system of claim 4, further comprising a first light source positioned to emit first light onto the first portion of the endoscope body,
wherein the insertion depth sensor defines a slit shaped to allow the first light to pass through to the first portion of the endoscope body.

7. The system of claim 6, further comprising a light baffle positioned between the first sensor and the first light source, wherein the light baffle is configured to block the first light from passing directly from the first light source to the first sensor.

8. The system of claim 4, wherein the first sensor and the second sensor are magnetic sensors positioned to generate insertion depth signals based on a magnetic field or a magnetic dipole moment; and wherein the alternating markings comprise:
a plurality of first bands having a first magnetic polarity; and
a plurality of second bands interspersed between first bands of the plurality of first bands, wherein the plurality of second bands has a second magnetic polarity different than the first magnetic polarity.

9. The system of claim 2, wherein the insertion depth sensor is shaped to remain at an insertion point in the portion of the body and allow the endoscope body to pass through the aperture into and out of the portion of the portion of the body.

10. The system of claim 2, wherein the insertion depth sensor is a trocar.

11. The system of claim 2, wherein the insertion depth sensor is an optical flow sensor.

12. The system of claim 1, wherein the insertion depth sensor comprises a plurality of capacitive sensors disposed along the endoscope body, wherein a capacitive sensor of the plurality of capacitive sensors is configured to generate a capacitive insertion depth signal when the capacitive sensor is inserted within and in contact with the portion of the body.

13. The system of claim 1, wherein generating positional information of the distal end of the endoscope body within the portion of the body comprises generating a three-dimensional map of the portion of the body based on a path the distal end has travelled within the portion of the body.

14. The system of claim 13, further comprising a user interface configured to receive input from a user to annotate the three-dimensional map based on the input from the user.

15. The system of claim 14, wherein the controller includes logic that, when executed, causes the system to perform operations including:
generating a marker signal when the distal end is located in a portion of the path corresponding to an annotated portion of the three-dimensional map.

16. A method of determining a position of a distal end of an endoscope in a portion of a body, the method comprising:
measuring an insertion depth of the endoscope in the portion of the body;
measuring an orientation of the distal end of the endoscope; and
determining the position of the distal end of the endoscope in the portion of the body based upon the measured insertion depth of the endoscope and the measured orientation of the distal end of the endoscope.

17. The method of claim 16, wherein
measuring the insertion depth of the endoscope in the portion of the body comprises generating, with an insertion depth sensor, an insertion depth signal based on the insertion depth of the endoscope within the portion of the body;
measuring the orientation of the distal end of the endoscope comprises generating, with an inertial measurement unit disposed at the distal end of the endoscope, an orientation signal based upon an orientation of the inertial measurement unit; and
determining the position of the distal end of the endoscope comprises generating positional information of the distal end of the endoscope body within the portion of the body based upon the insertion depth signal and the orientation signal.

18. The method of claim 17, wherein generating positional information of the distal end of the endoscope body within the portion of the body comprises generating a three-dimensional map of the portion of the body based on a path the distal end has traveled within the portion of the body.

19. The method of claim 18, further comprising generating an annotation signal based on a user input received from a user interface to annotate a portion of the three-dimensional map.

20. The method of claim 19, further comprising generating a marker signal when the distal end is located in the portion of the path corresponding to the annotated portion of the three-dimensional map.
